(19) United States
(12) Patent Application Publication
(10) Pub. No.: US 2009/0148485 A1
(43) Pub. Date: Jun. 11, 2009

(54) Controlled Release Devices and Structural Elements for Use in Their Manufacture

(75) Inventor: Derek James Whitehead, Cheshire (GB)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Castex Products Limited, New Mills Stockport (GB)

(21) Appl. No.: 11/918,380

(22) PCT Filed: Oct. 4, 2006
(86) PCT No.: PCT/GB2006/001327

§ 371 Date: Oct. 30, 2008

(30) Foreign Application Priority Data
Apr. 12, 2005 (GB) 0507383.8

(51) Int. Cl. A61F 13/00

(52) U.S. Cl. 424/422

(57) ABSTRACT

A structural element (2), suitable for use in the manufacture of a release device for intraruminal administration of an active agent to a ruminant animal, is of a compacted material, which material comprises a mixture of powdered iron, graphite and optionally copper, the graphite being present in the mixture in an amount of from 2 to 7 wt %, the copper in an amount of from 0 to 5 wt % and the iron in an amount of from 88 to 98 wt %, by weight of the total weight of iron, copper and graphite. A plurality of structural elements (2) may be assembled to provide a structural unit (4) of a release device.

The invention relates to structural elements suitable for use in the manufacture of controlled release devices, especially controlled release boli, and to such release devices for administration of active agents to ruminant animals. Such intraruminal boli are commonly employed to deliver active agents to ruminant animals over extended periods of time.

The invention also relates to the use, in the manufacture of controlled release boli, of such structural elements, which are capable of retaining their integrity so as to carry the active agent during the controlled release.

As may be seen from the following examples, current bolus designs are complex, leading to high component production and assembly costs.

Thus, EP-A-0164927 discloses a pulse release bolus for cattle which has gained wide acceptance and is based on the galvanic corrosion of magnesium as a timing mechanism. A longitudinal section of such a device is illustrated in FIG. 1. The device consists of a series of segments [a] of plastics material containing anthelmintic oxfendazole tablets [b] carried on a magnesium alloy support rod [c]. The bolus is weighted by a dense iron mass [j] to avoid regurgitation by the animal. A blank segment [d] provides an initial delay after administration and before an active tablet is released. The junctions between adjacent segments and the weight are sealed with rubber washers [e]. Typically, the device has a length of 90 mm and a diameter of 25 mm. The device operates by the progressive shortening of the magnesium support rod in the rumen fluid which results in the periodic discard of segments and release of active tablets. The bolus contains 19 separate components of 6 types made from 5 different constructional materials.

EP-A-0243111 discloses a continuous release bolus for cattle, also based on the corrosion of magnesium as a timing mechanism and widely used. A longitudinal section of such a device in accordance with EP-A-0243111 is illustrated in FIG. 2. The device consists of a series of tablets [f] inserted into a magnesium alloy support tube [g] the surface of which, apart from the extreme ends, is protected from attack by the rumen contents by close fitting, interlocking rings [h] of plastics material. Typically, the device has a length of 80 mm and a diameter of 28 mm. The tablets contain the active agent fenbendazole as an anthelmintic, graphite to provide a cathode to the magnesium and disseminated iron particles which serve to weight the bolus, together with binders and soluble agents. The bolus support tube corrodes from both ends, continuously releasing fenbendazole from the erodable tablets [f]. The bolus contains 21 separate components of 6 types made from 3 different constructional materials all produced to close dimensional tolerances.

Smaller devices similar to the above examples may be constructed for sheep.

With reference to EP-A-0243111 (supra), it is to be noted that, as illustrated by this document, iron and graphite combinations have been utilised in bolus manufacture for several years. However, in the manufacture of such degradable (erodable) parts of such boli, the graphite powder, in combination with iron shot particles, sugar and anthelmintic, is used to form a degradable tablet which is cathodic to an enclosing magnesium shell. The function of the graphite is to make the tablet electrically conductive and provide the electrode to promote galvanic corrosion of the magnesium shell. The rumen contents serve as the electrolyte. The iron is present in the form of coarse particles and acts solely as a weighting agent to avoid regurgitation of the bolus by the animal and does not contribute to the strength and integrity of the tablets or bolus. Other degradable tablets containing iron and graphite and used as the central core in bolus construction are described in EP-A-0482947 and WO-A-98/56347.

As will be explained more fully below with reference to the invention, such use as described above of iron and graphite in an erodable composition is in stark contrast with the use of these materials in producing a structural element carrying active and degradable components.

WO-A-96/01619 discloses a bolus for delivery of pulsed doses of biologically active material to a ruminant animal which comprises a series of distinct and separate structural elements, housed within an outer tube of an easily degradable material such as paper, and each defining a respective segment of the bolus. Each element is in the form of a short solid cylinder having a central recess in an axial end thereof, which recessed cylinder defines a dense cathodic sheath for a tablet of active material covered by a plug of magnesium each within the recess. Degradation of the outer tube releases the individual segments, which disperse within the rumen. Galvanic corrosion of the magnesium plug under the influence of the cathodic sheath then releases the tablet. The cathodic sheath may be formed by compacting a 50-50 mixture (by volume) of pure iron and graphite powders, thus providing an iron/graphite mixture containing about 13 wt % graphite, by weight of the mixture. However, as described there, compacting requires a load as high as 10 tonnes/cm². Moreover, even then, physical properties such as rupture load and water repellancy are not satisfactory.

In the case of cattle with internal parasites the live weight gain of the animal due to the use of boli can more than compensate for the cost of treatment and there is a strong economic case for their use. This may apply particularly to some regions of the Western Hemisphere where the value of the animals is higher than elsewhere. However for sheep the cost saving may be less attractive due to the lower value of the animal. Boli which give extended protection against parasites e.g. for periods which may exceed four months by pulse or continuous release of the active agent are, as illustrated above, generally complex assemblies of several different types of component. Although lower doses of active agent are required for sheep and the boli are smaller and use less materials than those for cattle the component production and assembly costs are much the same for both types of bolus and any savings are generally inadequate to compensate for the lower value of the animal. There is hence a need for less complex and lower production cost devices and this is particularly the case for sheep. Again, there is little doubt that intraruminal anthelmintic boli would find increased use in Southern Hemisphere countries, where animal values are lower, if cheaper variants were available.

The present invention seeks to address the drawbacks and limitations of conventional release devices discussed above.

We have now found surprisingly that, if a bolus has a structural unit compacted from a mixture of iron, graphite and optionally copper but with lower proportions of graphite in the mixture than that of the release devices disclosed in WO-A-96/01619, then both improved performance and ease of manufacture can be achieved.

Moreover, the use of such a structural unit in the manufacture of a release device allows the production of a preferred release device in accordance with the invention in which segments of the bolus are released one at a time for progressive sustained release of active material from respective segments.

According to a first aspect, the present invention provides a structural element suitable for use in the manufacture of a release device for intraruminal administration of an active agent to a ruminant animal, which structural element is of a compacted material, which material comprises a mixture of powdered iron, graphite and optionally copper, the graphite being present in the mixture in an amount of from 2 to 7 wt %, the copper in an amount of from 0 to 5 wt % and the iron in an amount of from 88 to 98 wt %, by weight of the total amount of iron, copper and graphite.

The structural element is particularly suitable for use as a segment of a structural unit comprising an assembly thereof.

The structural element may have a radial cross section which is circular and, in particular, may have a cylindrical configuration especially suitable for a cylindrical bolus.

The structural element preferably comprises a cylindrical element having a central cylindrical aperture therein, coaxial with the structural element and adapted to receive, with an interference fit, a core of anodic material.

At least one end face of the structural element may have a recess therein adapted to receive a dosage form of an active agent composition. Indeed, at least one end face may have a plurality of recesses, typically from 2 to 10, each adapted to receive a respective said dosage form. For example, a plurality of circular section recesses may accommodate a plurality of circular section tablets.

The recess or recesses may be of any desired shape in order to accommodate any desired shape of dosage form and may be of any volume sufficient to accommodate a pulsed dose of active agent. For example, a circular section recess is suitable for accommodating a correspondingly shaped tablet in the form of a disc while an annular recess is suitable for receiving a ring shaped tablet. The recess may be located in any position in the end face but is preferably spaced from each of the internal and external peripheries of the structural element. However, a ring shaped tablet may be accommodated within a circular section recess coaxial and radially coextensive with an aperture adapted to receive a core of anodic material as described above. However, the inner diameter of the ring shaped tablet must then be sufficiently wide to avoid significant contact with the core during assembly of the bolus. As described below, when one or more recesses are provided on each of opposed faces, recesses in adjacent faces of adjacent structural elements may cooperate to hold a single dosage form.

A plurality of structural elements as described above may be assembled to provide a structural unit, preferably of cylindrical form.

Such a structural element may have at least one recess in each of opposed end faces thereof, whereby, in the assembly of structural elements, the or each recess in an end face of a structural element being in register with a corresponding recess in an adjacent end face of an adjacent structural element, respective corresponding recesses thereby cooperating with one another to house a single dosage form.

A structural element in accordance with the first aspect of the invention may be prepared by compacting, by, for example, pressing or moulding, the material comprising the mixture of powdered iron, graphite and optionally copper.

The powders to be compacted preferably have a mesh size, as measured by British Standard (BS) 410, within the range 100-400 mesh, more preferably 300 mesh.

The compaction of the material comprising powdered graphite, iron and any copper may result in a release device having a density of 3-6.5 gm/ml. This density depends upon the pressure applied and the proportional amount of graphite and copper present.

In a preferred embodiment, the material of the structural element consists of a mixture of iron, in an amount of from 88 to 98 wt %, copper, in an amount of 0 to 5 wt % and graphite, in an amount of 2 to 7 wt %, by weight of total material.

In a more preferred embodiment, the amount of copper in the structural element is at least 1 wt %, still more preferably from 1 to 4 wt %, especially about 3 wt %. The addition of copper may provide a harder compacted material and may reduce the tonnage required for compaction.

The structural element material may comprise a mixture which additionally includes a lubricant, for example, magnesium stearate. If present, the amount is then preferably up to 2 wt %, more preferably up to 1 wt %, based on the total weight of the material.

With reference to the proportional amount of graphite, if the amount is too low, adequate lubricity and freedom from moisture penetration of the structural units by the rumen contents may not be guaranteed. If the amount is too high, the structural unit will become too fragile.

With respect to the manufacture, and ultimate performance in a release device, of a structural element embodying the invention, the iron, graphite and copper have the following influences.

### Iron

(a) Strength—aids assembly, prevents failure.
(b) Density—sufficiently high to avoid regurgitation.
(c) Electrode Potential—enhances corrosion.
(d) Mouldability—can be pressure formed from powder.
(e) Cost—it is inexpensive.

### Graphite

(a) Processing—enhances mouldability.
(b) Lubrication—no additional lubricant normally needed for moulding.
(c) Porosity—heals porosity so that the segments do not absorb water.
(d) Water repellant—very high so that the water does not wet and penetrate the segments.
(e) Electrical conductivity—provides sufficient electrical conductivity or, in any event, does not reduce the conductivity of iron and copper.
(f) Assembly—improves physical properties, especially shear strength.

### Copper

(a) Strength—augments strength conferred by iron.
(b) Mouldability—enhanced by copper.
(c) Cost—it is relatively inexpensive.

From the above, it can be seen that, in contrast with the bolus construction of EP-A-0243111, described previously, where the iron serves only as a weighting agent so as to avoid regurgitation by the animal, here it performs simultaneously the functions of a weighting agent and a cathodic element and also provides the required strength. Moreover, when copper is present this further augments the strength of the bolus.

Similarly, in contrast with the bolus disclosed in WO-A-96/01619, also comprising structural elements made up of iron and graphite, the structural element in accordance with the invention, which contains substantially lower proportions of graphite (2-7 wt %) as compared with that of the structural elements of WO-A-96/01619 (about 13 wt %), it is possible to compact the material into pressings having the desired physical properties such as rupture load; the physical properties of pressings from the material of WO-A-96/01619 are so inferior to those of a structural element of the invention as to render them unsuitable for fabrication into a bolus in accordance with the invention.

According to a second aspect, the present invention provides a release device for intraruminal administration of an active agent such as a medicament or dietary supplement, to a ruminant animal, the device having a degradable member, a non-degradable structural unit and an active agent composition carried by the non-degradable structural unit, which structural unit is made from a compacted material, which material comprises a mixture of powdered iron, graphite and optionally copper, the graphite being present in the mixture in an amount of from 2 to 7 wt %, the copper in an amount of from 0 to 5 wt % and the iron in an amount of from 88 to 98 wt % of the total weight of iron, copper and graphite, whereby the compacted material is capable of enhancing the density of the release device and capable of promoting galvanic corrosion, by rumen juices, of the degradable member, the degradable member and non-degradable structural unit being arranged so that intraruminal corrosion of the degradable member exposes the non-degradable structural unit and active agent composition carried thereby, thus enabling release of the active agent into the rumen juices.

According to a third aspect, the invention provides the use, in the manufacture of a release device for intraruminal administration of an active agent to a ruminant animal, of a non-degradable structural unit of a compacted material, which material comprises a mixture of powdered iron, graphite and optionally copper, the graphite being present in the mixture in an amount of from 2 to 7 wt %, the copper in an amount of from 0 to 5 wt % and the iron in an amount of from 88 to 98 wt % of the total weight of iron, copper and graphite, whereby the compacted material is capable of enhancing the density enhancing component and a component capable of promoting galvanic corrosion, by rumen juices, of a degradable member of the release device.

In a preferred release device in accordance with a second aspect of the invention, the non-degradable structural unit thereof preferably comprises at least one structural element of the first aspect of the invention and more preferably comprises an assembly of such structural elements used as respective segments of a structural unit.

A release device in accordance with a second aspect of the invention is particularly suitable for the treatment of cattle and sheep with anthelmintics for the control of intestinal parasites which represents a major share of the animal health market for cattle and sheep but it may also be used with other active agents including therapeutics and nutrients. The release device is preferably an intraruminal bolus where the active agent release mechanism is determined by the corrosion of magnesium by the rumen fluid.

Thus, preferably the degradable member comprises magnesium, more preferably, a magnesium alloy. Still more preferably, the degradable member is a magnesium alloy core, which may be for example, rod-shaped or tubular, as known from the prior art.

The magnesium alloy preferably contains at least 70 wt % of magnesium and any one or more of aluminium (preferably up to 15 wt %), zinc (preferably up to 5%), copper (preferably up to 4 wt %), manganese (preferably up to 2 wt %), silicon (preferably up to 1.5 wt %), and zirconium (preferably up to 0.8 wt %).

In a preferred release device embodying the invention, in which a plurality of segments of the non-degradable structural unit surround a degradable core, the structural unit serves several functions simultaneously, namely
(a) it provides a support for dosage forms of the active agent;
(b) it serves to prevent ingress of rumen juices into the release device until degradation of the degradable core;
(c) it confines corrosion of the degradable core to regions not surrounded by the structural unit;
(d) it enhances the density of the release device to prevent regurgitation by the animal; and
(e) it promotes galvanic corrosion of the degradable core.

Thus, as previously mentioned, unlike certain prior art release devices (see, for example, EP-A-0243111, supra) where the iron serves only to add weight to the device, in contrast, in a release device embodying the invention, the iron is also incorporated in a structural unit, which structural unit simultaneously confers strength upon the device, provide a cathode for promoting galvanic corrosion of the anode and provides a carrier for a dosage form of the active agent. This has the advantage that no further components are needed to provide the desired compressive strength and structural integrity, for example an outer layer of plastics material is not needed, sealing washers between respective segments are not needed and the degradable component can be a core, rather than a tube. This simplified approach reduces the complexity of the device and its manufacture, and reduces costs.

Preferably, the release device includes a structural unit comprising a plurality of segments in face to face relation with one another, including segments having, in at least one axial end face thereof, a recess capable of receiving a dosage form of the active agent composition.

Suitably, the segments are sequentially released into the rumen juices as the degradable member degrades. The segments may be interlinked to assist in preventing the ingress of moisture into the device. Alternatively or additionally the segments may simply abut one another within the device.

In a first preferred embodiment, each of a plurality of adjacent segments in face to face relation with one another has one axial end face having at least one recess in face to face relation with an axial end face of an adjacent segment free from a recess.

In an alternative, second preferred embodiment, each of adjacent segments in face to face relation with one another has at least one recess in each axial end face, each recess in an axial end face lying in register with a corresponding recess in an adjacent end face of an adjacent segment, whereby respective adjacent opposed recesses together define a cavity in which sits a dosage form. The dosage form therefore contains twice the dosage of a dosage form in a single recess.

By increasing the length of each segment, this second embodiment allows a greater time interval between albeit double dosages. Thus, such a double dose could be given less frequently for example, at 40-day as opposed to 20-day intervals. In this way, the time taken for certain parasites to become immune to the drug if administered too often can be delayed.

A similar effect may be achieved using structural elements in accordance with the above first embodiment as segments in an assembly thereof in which adjacent segments were arranged so that those respective axial end faces having at least one recess therein lay in face to face relation with one another such that respective adjacent opposed recesses in register with one another together defined a cavity for a double dosage form, while those respective axial end faces free from respective recesses also lay in face to face relation with one another so as to provide a longest length between adjacent faces containing recesses.

Suitably, the non-degradable structural unit is arranged so that it protects a large proportion of the total peripheral surface of the degradable member from the rumen juices. Since the non-degradable structural unit does not degrade in the rumen juices there is no need to provide a separate protective layer of plastics material or resin. Preferably, the degradable member is a core and the non-degradable structural unit is arranged around the core to protect the peripheral surfaces of the core.

Both ends of the core may be uncovered so that they are exposed to the rumen juices when the device is administered. In such a case, the peripheral surface between the ends may be entirely covered by the structural unit. Alternatively, one of the longitudinal ends of the core may also be covered, either by a segment of the structural unit, shaped so as to provide a covering element, or simply by means of a plug.

The structural unit preferably comprises a plurality of annular section segments having respective central apertures together defining a central aperture of the structural unit and the degradable member is a solid cylindrical rod making an interference fit with the central aperture of the structural unit.

The active agent may be provided in an active agent composition. Preferably the active agent composition is in dosage, for example, tablet, form.

A particularly preferred release device in accordance with the second aspect of the invention is a sustained release bolus comprising
a structural unit comprising a plurality of annular segments in face to face relation with one another and having respective central apertures together defining a central cylindrical aperture of the structural unit;
a central cylindrical core of a magnesium alloy making an interference fit within the central cylindrical aperture of the structural unit;
the structural unit including segments having, in at least one axial end face thereof, at least one recess; and
a dosage form of a medicament housed in each respective recess of the segments;
each annular segment comprising a compacted material, which material comprises a mixture of powdered iron, graphite and optionally copper, the graphite being present in the mixture in an amount of from 2 to 7 wt %, the copper in an amount of from 0 to 5 wt % and the iron in an amount of from 88 to 98 wt %, by weight of the total weight of iron, copper and graphite, which compacted material is capable of enhancing the density of the bolus promoting galvanic corrosion of the magnesium alloy core; and
at least one axial end region of the core being exposed to allow the said galvanic corrosion thereof.

In contrast with the construction of release device shown in WO-A-96/01619, which provides for release of the segments for dispersion within the rumen, the above release device embodying the invention provides release of the segments one at a time.

This construction embodying the invention allows more accurate estimation of the time of release of the medicament and avoids the formation of an unwanted oxide layer between the magnesium and the graphite.

Segments of the structural unit comprising a compacted mixture of iron and graphite may be made simply and economically as small pressings of the mixture of iron, graphite and, when present, copper.

Each segment may have a length of from 5 to 20 mm so as to provide a structural unit having a total length of about 20-100 mm, especially 60-80 mm, typically 70 mm. Thus, for treatment of cattle, a typical length of structural unit may be from 15 to 25 mm, especially 19-21 mm, while for treatment of sheep, a typical length may be from 10 to 20 mm, especially 14-16 mm. In each case, a diameter of from 15 to 25 mm, especially 18-20 mm, is suitable for both cattle and sheep. The segment length is determined by the required pulse interval, which intervals need not always be equal. Thus, it is possible to include, within a given release device, segments of different respective lengths.

Preferably, the pressure under which the iron-graphite member is compacted is at least about 5 tonnes/cm² and more preferably at least about 6 tonnes/cm². An upper limit of pressure is about 10 tonne/cm². Suitably, the pressure is in the range of about 6 tonne/cm² to about 9 tonne/cm². A pressure of about 7-8 tonne/cm² is particularly preferred.

The term ‘non-degradable’ as used herein with reference to the material of structural units and segments thereof means that the material in question does not degrade in rumen juices on a timescale relevant to the administration of the active agents described herein. The non-degradable materials are therefore preferably resistant to degradation by chemical, biological or physical processes that might be experienced when administered to a ruminant.

### BRIEF DESCRIPTION OF THE DRAWINGS

Embodiments of the invention will now be described, by way of example only, with reference to the accompanying drawings of such embodiments and, by way of comparison, drawings of conventional boli. In the drawings (where FIGS. 1 to 4 are all in longitudinal section, while FIG. 5 is a schematic illustration):

FIG. 1 shows a first conventional bolus in accordance with EP-A-0164927, as has already been discussed;

FIG. 2 shows a second conventional bolus in accordance with EP-A-0243111, again as previously discussed;

FIG. 3 shows a release device, being a first embodiment of the present invention;

FIG. 4 shows a second release device, being a second embodiment of the present invention; and

FIG. 5 shows a third release device, being a third embodiment of the present invention.

### DETAILED DESCRIPTION OF EMBODIMENTS AND EXPERIMENTS

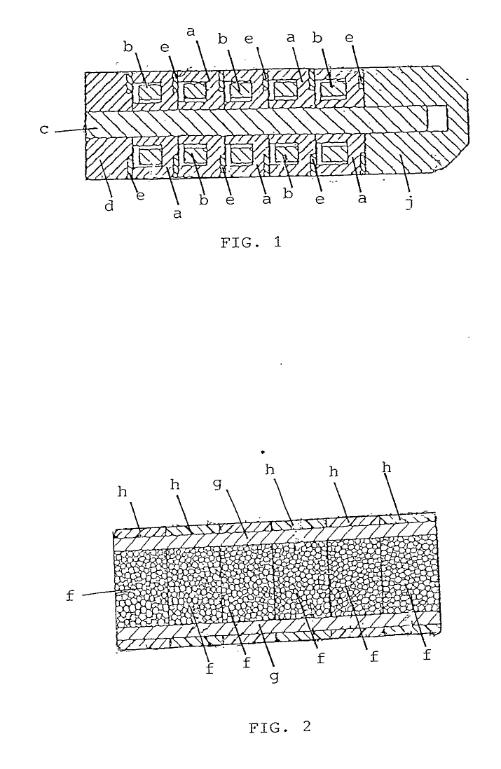

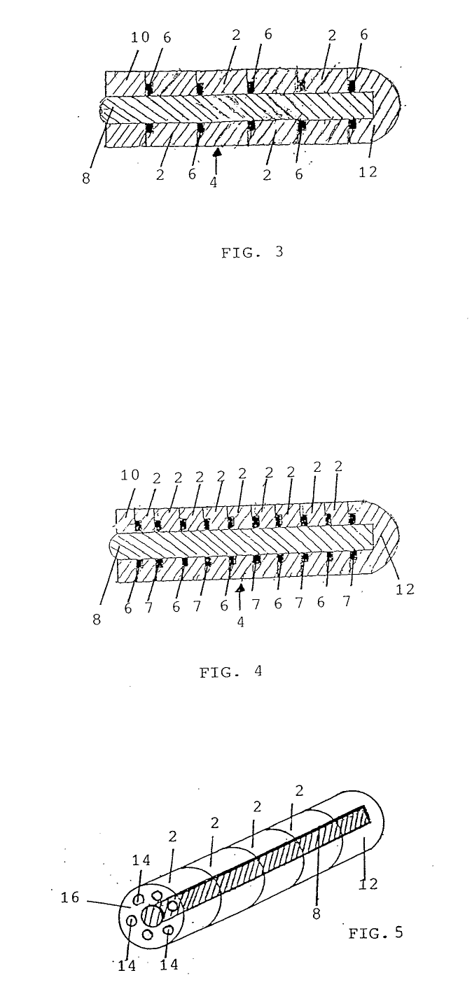

The simplicity of bolus designs embodying this invention may be seen from the longitudinal section of the bolus shown in FIG. 3. Here, structural elements, in the form of compacts of iron, graphite and optionally copper, including respective segments [2] of a structural unit, generally indicated by arrow [4], carrying active tablets [6], are supported on a magnesium alloy rod [8]. A blank structural element [10] provides an initial delay after administration and before an active tablet [6] is released, while a structural element [12], in addition to carrying a tablet, is profiled so as to provide a covering element. The release mechanism is similar to that of the bolus shown in FIG. 1 in that dissolution and shortening of the rod by galvanic action in the rumen fluid results in the progressive discard, firstly of the blank structural element [10], and then of the segments [2] and release of the active tablets [6] containing, for example, ivermectin. Typically, the bolus of FIG. 3 has a length of 70 mm and a diameter of 19 mm.

FIG. 4 illustrates a further and more complex device suitable for the delivery of multi actives, particularly for sheep, where resistance problems to the benzimidazoles and ivermectin are being increasingly encountered. Here the segments [2], of iron, graphite and optionally copper hereinafter referred to as “iron-copper-graphite segments [2]” are approximately half the length of those shown in the bolus of FIG. 3. A tablet is hence released at approximately 10 day intervals. The bolus contains for example three actives, benzimidazole and ivermectin in tablets [6] and levamisole in tablets [7]. Trace elements such as cobalt and selenium in the form of soluble compounds may also be incorporated in the active tablets. Each drug is released at 20 day intervals and the active content of the bolus is twice that of the bolus illustrated in FIG. 3. This is gained at the expense of a more complex assembly operation but little difference in segment cost due to the high rate of production. Again, typically, the bolus of FIG. 4 has a length of 70 mm and a diameter of 19 mm.

FIG. 5 is a schematic illustration of yet another release device embodying the invention in which, in place of the single recess for a ring shaped tablet dosage [6] as provided in the device of FIGS. 3 and 4, here, a plurality of recesses [14], each spaced from the central core, are provided in each iron-copper-graphite segment [2] for respective disc-shaped tablets. The bolus of FIG. 5 has a closed end provided by a covering element [12] and an open end [16], so that on administration to an animal of a tablet dosage is immediately released into the animal.

The economy and simplicity of the devices shown in FIGS. 3 to 5 is achieved by the use of a novel iron-graphite segment which has the following advantages of ease and speed of manufacture and economy of materials, resulting in simplicity of bolus assembly and flexibility of bolus design.

### Manufacture of Structural Unit Segments.

A uniform blend of fine, e.g. 300 mesh [BSS 410], iron, graphite and optionally copper powders is readily prepared in any of a variety of industrial and pharmaceutical mixers. In general, the powder mix has little tendency to segregate and can be pressed into segments at high speed on a rotary tablet machine. Very high rates of output can be achieved e.g. 70,000 segments per hour. In contrast a typical 50 tonne capacity injection moulding machine will produce about 600 segments of plastics material similar to those shown in FIG. 1 per hour. For a given bolus output, which may amount to several million per year, substantial savings may be made in production and capital investment costs. Energy savings are particularly significant.

In cases where the strengths of segments produced on rotary machines are inadequate, due either to limitation of the loads that may be applied by particular presses or the ability of the tooling used to withstand the stresses involved, then recourse may be had to heat treatment of the segments. Standard sintering procedures such as are commonly used with iron powder pressings may be applied to the iron-copper-graphite segments of this invention. Substantial improvements in segment strengths may be achieved. However, this is gained at the expense of increased permeability to water penetration, loss of lubricity and increased cost. Heat treatment in steam atmospheres is also applicable at temperatures in excess of about 400° C.

### Economy of Materials.

The segments, shown in FIG. 1, of plastics material are made from high grade unplasticised polyvinyl chloride, a material with good physical and chemical properties for bolus use. However, the yield of segments is typically less than 70% of the material processed. This is due to degradation of the plastics material at the moulding temperature and inability to recycle process scrap. In contrast the yield of segments from iron-graphite powder can exceed 95%. Although iron-graphite is more expensive on a volume basis than polyvinyl chloride its high density means that the weight [j] shown in FIG. 1 of the conventional bolus can be dispensed with, offsetting the volume price advantage of the plastics material. Similarly the washers [e] sealing the junctions between adjacent segments are not required for iron-graphite due to the water repellant nature and high degree of dimensional accuracy and quality of surface finish of the latter.

### Simplicity of Bolus Assembly.

Reductions in the number and types of component lead to an automated assembly process using less complex and fewer machines with production time and capital cost savings.

Thus, a preferred bolus in accordance with the invention, in which the structural unit comprises a plurality of structural elements each having a central aperture and including segments carrying tablets of active agent composition, can be manufactured simply by providing the tablets in respective recesses in axial end faces of the segments, stacking the structural elements one above the other in axial alignment in a suitable housing so as to assemble a structural element having a central aperture and pressing a core of degradable material into the central aperture of the structural unit with an interference fit to obtain a bolus.

### Flexibility of Bolus Design.

The interval between release of doses of active agent, e.g. in tablet form, can be readily changed by altering the length of the iron-copper-graphite segments. Additional methods including varying the characteristics of the degradable member, for example, where this is a magnesium alloy support rod, adjusting the diameter of the rod and changing the alloy composition. Data contained in WO-A-98/56347 illustrates how the alloy composition may be changed.

### Active Tablet Manufacture

This follows standard pharmaceutical practice. A variety of binders, lubricants and bulking agents including polyvinylpyrrolidone, starch, magnesium stearate and lactose may be included.

### Graphite Content

The concentration of graphite in an iron-graphite segment of a structural unit affects the properties of the segment material. Graphite provides lubricity to the non-degradable component and also helps prevent moisture penetration of the segments by the rumen contents.

Thus, very low graphite concentrations, especially less than about 2 wt %, may not be desirable because concentrations below this level may provide insufficient lubricity and protection against ingress of moisture.

Similarly, high graphite concentrations are less desirable because high concentrations, e.g. more than about 7 wt %, reduce the strength of the iron-graphite segment. High graphite concentrations may also reduce the density of the device to an extent where the density is no longer sufficient to prevent regurgitation of the device without the addition of supplementary weights.

It is not desirable for the non-degradable member to be made entirely from iron powder due to problems with ingress of water caused by porosity.

The porosity of the non-degradable member can be reduced by increasing the pressure applied to the iron-graphite mixture when it is compacted. Increased pressure also increases the strength of the non-degradable member. Generally, the upper limit for the pressure that can be applied to the iron-graphite mixture, and hence the strength of the non-degradable member, is governed by the maximum capacity of the rotary tablet press to apply force (pressure) to the mixture. Typically the maximum force that a conventional rotary tablet press can apply is about 20 tonnes. Higher forces may be applied, and hence potentially higher component strengths achieved, if single stroke presses are used. However, productivity is then much reduced.

Table 1 gives the results of a series of transverse rupture tests carried out on solid, circular section, iron-graphite discs having varying concentrations of graphite. Such tests, carried out on a simpler, solid construction, give more reliable results than a test carried out on a more complex, annular construction of segment in accordance with a preferred embodiment of the invention. Moreover, they still allow the behaviour of a corresponding segment in dependence upon graphite concentration to be predicted. The discs were formed by mixing 300 mesh iron and graphite powders as discussed above, and then pressing them. The disc size was 26 mm diameter×6 mm thick. A preferred iron-graphite composition for compacting into a structural element in accordance with the invention can preferably withstand a transverse rupture load of at least, and more preferably in excess of, 200 kg when pressed into the above-mentioned form of a 26×6 mm solid disc.

TABLE 1

| | Pressure | Segment | Rupture Load |
|---|---|---|---|
| Graphite wt % | tonne/cm² | density g/cm³ | kg |
| 0 | 3.8 | 6.3 | >245* |
| 1 | 3.8 | 6.2 | >245 |
| 2 | 3.8 | 6.2 | >245 |
| 3 | 3.8 | 6.2 | 243 |
| 4 | 3.8 | 6.2 | 210 |
| 5 | 3.8 | 6.1 | 175 |
| 7 | 3.8 | 5.8 | 179 |
| 10 | 3.8 | 5.6 | 77 |
| 15 | 3.8 | 5.2 | 95 |
| 100 | 3.8 | 2.1 | 12 |
| 0 | 6.6 | 6.5 | >245 |
| 1 | 6.6 | 6.5 | >245 |
| 2 | 6.6 | 6.5 | >245 |
| 5 | 6.6 | 6.5 | 240 |
| 7 | 6.6 | 6.4 | 216 |
| 10 | 6.6 | 6.5 | 144 |
| 100 | 6.6 | 2.0 | 36 |

*Maximum capacity of testing machine

As can be seen from the tabulated results, the relative proportions of iron and graphite in the powder mix can be adjusted to provide the non-degradable segments with adequate strength to survive the bolus assembly stresses without failure. In embodiments having a magnesium alloy support rod, insertion of this through a central aperture in the segments can impose stresses on the iron-graphite segments which can cause them to crack if the interference fit between the rod and the segments is too great for the strength of the compacted iron-graphite material.

In manufacture, the degree of variation of the interference fit is governed primarily by the tolerance on the rod diameter. With good production practice this can preferably be limited to e.g. +/−0.01 mm on a typical 7 mm diameter rod.

Tests have indicated that segments of the type shown in FIG. 3 should be pressed at loads in excess of 6 tonnes/cm², based on projected area, to ensure freedom from cracking when rods are inserted with a low tolerance interference fit.

The transverse rupture tests listed in Table 1 show that a reduction of segment strength may occur where the graphite concentration exceeds about 7 wt %.

### Copper Content of Iron-Graphite-Copper Segment Pressings.

Copper powder is a useful component of segments according to the present invention. It significantly increases the strength of the compacts. Table 2 gives the results of transverse rupture tests carried out, in the manner described with reference to Table 1, on solid 26 mm diameter×4.7 mm thick iron-graphite powder compacts containing 5 and 7 wt % of graphite with an addition of 3 wt % copper. To bring the recorded strengths within the range of the tablet testing machine the thickness of the tablets was reduced from the 6 mm employed with the tests of Table 1 to 4.7 mm. The test results illustrate the significant strengthening effect of a 3 wt % copper powder addition to the iron-graphite tablets.

TABLE 2

| | Pressure | | Rupture Load |
|---|---|---|---|
| Graphite wt % | tonne/cm² | Copper wt % | Kg |
| 5 | 3.8 | 0 | 136 |
| 5 | 3.8 | 3 | 150 |
| 7 | 6.6 | 0 | 171 |
| 7 | 6.6 | 3 | 188 |

1. A structural element suitable for use, as a segment of a structural unit comprising an assembly of the structural elements, in the manufacture of a release device for intraruminal administration, to a ruminant animal, of an active agent housed in the structural unit, which structural element is of a compacted material, which material comprises a mixture of powdered iron, graphite and copper, the graphite being present in the mixture in an amount of from 2 to 7 wt %, the copper in an amount of up to 5 wt % and the iron in an amount of from 88 to 98 wt %, by weight of the total weight of iron, copper and graphite, and which structural element has, in at least one end face thereof, a recess, adapted to receive a dosage form of the active agent, and has, passing through the structural element, a central aperture adapted to receive a core of anodic material.

2. A structural element according to claim 1, wherein the compacted material consists of a mixture of powdered iron, copper and graphite.

3. A structural element according to claim 1, wherein the compacted material contains at least 1 wt % copper, based on the total weight of the compacted material

4. A structural element according to claim 1, wherein the amount of copper in the structural element is from 1 to 4 wt % by weight of total material.

5. A structural element according to claim 1, having in at least one end face thereof a plurality of recesses, each adapted to receive a respective said dosage form.

6. A structural element according to claim 1, having at least one recess in each of opposite end faces thereof, whereby, in the said assembly of structural elements, the or each recess in an end face of a structural element is in register with a corresponding recess in an adjacent end face of an adjacent structural element, respective corresponding recesses thereby cooperating with one another to house a single dosage form.

7. A structural element according to claim 1, having a radial cross section which is circular.

8. A structural element according to claim 7, which is cylindrical.

9. A structural element according to claim 7, wherein the central aperture therein is a cylindrical aperture, coaxial with the structural element and adapted to receive, with an interference fit, a cylindrical core of anodic material.

10. A release device for intraruminal administration of an active agent to a ruminant animal, the device having a degradable member, a non-degradable structural unit and an active agent composition carried by the non-degradable structural unit, which structural unit is of a compacted material, which material comprises a mixture of powdered iron, graphite and copper, the graphite being present in the mixture in an amount of from 2 to 7 wt %, the copper in an amount of up to 5 wt %, and the iron in an amount of from 88 to 98 wt %, by weight of the total weight of iron, copper and graphite, whereby the compacted material is capable of enhancing the density of the release device and capable of promoting galvanic corrosion, by rumen juices, of the degradable member, in which release device the degradable member has a central core surrounded by the structural unit, whereby a substantial proportion of the peripheral surface of the central core is protected, by the structural unit, from corrosion by rumen juices, and the structural unit comprises a plurality of segments in face to face relation with one another, each segment having a central aperture through which the central core passes and a plurality of adjacent said segments each having at least one recess receiving a dosage form of the active agent composition, at least one longitudinal end of the central core being open to corrosion, whereby progressive corrosion of the central core causes release from the device of successive segments of the structural unit and of the dosage form of the active agent associated with the said segments.

11. A release device according to claim 10, wherein the compacted material consists of a mixture of iron, copper and graphite.

12. A release device according to claim 10, wherein the compacted material contains at least 1 wt % copper, based on the total weight of the compacted material.

13. A release device according to claim 10, wherein the amount of copper in the structural element is from 1 to 4 wt % by weight of total material.

14. A release device according to claim 10, wherein the degradable member comprises a magnesium alloy.

15. A release device according to claim 10, wherein each of a plurality of adjacent segments in face to face relation with one another has one axial end face having at least one said recess in face to face relation with an axial end face of an adjacent segment free from a said recess.

16. A release device according to claim 10, wherein each of a plurality of adjacent segments in face to face contact with one another has at least one recess in each of opposite end faces thereof, the or each recess in an axial end face of a segment being in register with a corresponding recess in an adjacent axial end face of an adjacent segment, whereby respective corresponding recesses cooperate with one another to house a dosage form.

17. A release device according to claim 10, wherein segments in face to face relation with each other have, in at least one axial end thereof, a plurality of recesses each capable of receiving a dosage form of the active agent composition.

18. A release device according to claim 10, wherein the structural unit additionally comprises a covering element adapted to cover a longitudinal end of the central core opposite the said longitudinal end thereof to prevent exposure thereof.

19. A release device according to claim 10, wherein the structural unit comprises a plurality of annular section segments having respective central apertures together defining a central aperture of the structural unit and the degradable member is a solid cylindrical rod making an interference fit within the central aperture of the structural unit.

20. A sustained release bolus comprising
a structural unit comprising a plurality of annular segments in face to face relation with one another and having respective central apertures together defining a central cylindrical aperture of the structural unit;
a central cylindrical core of a magnesium alloy making an interference fit within the central cylindrical aperture of the structural unit;
the structural unit including segments having, in at least one axial end face thereof, at least one recess; and
a dosage form of a medicament housed in each respective recess of the segments;
each annular segment comprising a compacted material which material comprises a mixture of powdered iron, graphite and copper, the graphite being present in the mixture in an amount of from 2 to 7 wt %, the copper in an amount of up to 5 wt % and the iron in an amount of 88 to 98 wt %, by weight of the total weight of iron, copper and graphite, which compacted material is capable of enhancing the density of the bolus and promoting galvanic corrosion of the magnesium alloy core; and
at least one axial end region of the core being exposed to allow the said galvanic corrosion thereof.

21. A substantial release bolus according to claim 20, wherein the amount of copper in the annular segment is from 1 to 4 wt % by weight of total material.

22. (canceled)

23. Use, as a non-degradable structural element of a structural unit comprising an assembly of the structural elements in the manufacture of a release device for intraruminal administration, to a ruminant animal, of an active agent housed in the structural unit, which non-degradable structural element is made from a compacted material comprising a mixture of powdered iron, graphite and copper, the graphite being present in the mixture in an amount of from 2 to 7 wt %, the copper in an amount of up to 5 wt % and the iron in an amount of 88 to 98 wt %, by weight of the total weight of iron, copper and graphite, which compacted material is capable of enhancing the density and capable of promoting galvanic corrosion, by rumen juices, of a degradable member of the release device, and which structural element has, in at least one end face thereof, a recess, adapted to receive a dosage form of the active agent, and has, passing through the structural element, a central aperture adapted to receive a core of anodic material.

24. Use according to claim 23, wherein the compacted material consists of a mixture of iron, graphite and copper.

25. Use according to claim 23, wherein the compacted material contains at least 1 wt % copper, based on the total weight of the compacted material.

26. Use according to claim 23, wherein the amount of copper in the structural element is from 1 to 4 wt % by weight of total material.

27. (canceled)
